(19) United States
(12) Patent Application Publication
(10) Pub. No.: US 2020/0324075 A1
(43) Pub. Date: Oct. 15, 2020

(54) METHOD AND APPARATUS FOR MEDIATING PROPRIOCEPTIVE SKIN AND BODY SENSORS, VISION, VESTIBULAR AND SOMATO-SENSATION ACTING IN COMBINATION WITH BRAIN FUNCTION OR NEUROSENSORY-MOTOR SYSTEM FUNCTION

(71) Applicant: Motion Therapeutics, Inc., Oakland, CA (US)

(72) Inventor: Cynthia GIBSON-HORN, Oakland, CA (US)

(21) Appl. No.: 16/909,889

(22) Filed: Jun. 23, 2020

Related U.S. Application Data
(63) Continuation of application No. 14/781,523, filed on Sep. 30, 2015.
(60) Provisional application No. 61/807,260, filed on Apr. 1, 2013.

(51) Int. Cl. A61M 21/02; A61B 5/0484; A61B 5/00

(52) U.S. Cl. CPC A61M 21/02; A61B 5/0484; A61B 5/0048; A61M 2021/0016; A61B 5/4836; A61B 5/4023; A61B 5/6802

(57) ABSTRACT

An apparatus and method for improving functioning of at least a portion of a subject's nervous pathway system includes a garment or device worn or disposed on the subject; and at least one stimulator disposed on, in or within the garment or device capable of providing at least one corrective non-weight stimulus to the subject's nervous pathway system. The kind of nonweight stimulus provided by the stimulator to the subject's nervous pathway system, the amount, degree or intensity of the stimulus provided by the stimulator to the subject's nervous pathway system, and/or the body location of non-weight stimulus provided by the stimulator to the subject's nervous pathway system is determined by a reiterative diagnostic method leading to an optimal response for the treated subject.

### PRIORITY

This application is related to U.S. Provisional Patent Application Ser. No. 61/807,260, filed on Apr. 1, 2013, which is incorporated herein by reference and to which priority is claimed pursuant to 35 USC 119.

### BACKGROUND

### Field of the Technology

The disclosure relates to the field of sensory motor orthotic/prosthetic systems and methods for detecting and/or mediating proprioceptive loss, inability to engage the appropriate muscles (hypo or hypertonic), or decreased ability to interpret sensory information in a person, mammal or other animal having a postural control, balance, or other disorder with a stimulating device tending to improve one or more of the person's or animal's function; including visual, somatosensory, vestibular, perception of an upright posture, sway, static and dynamic balance, motor control coordination, gait, speech, swallowing, handwriting and/or motor control.

### Description of the Prior Art

Postural control and symmetry of the body of a human and animal is described as the ability to maintain a neutral position in static upright static positions, such as sitting or standing, during dynamic activities, such as moving from one position to another, and to react to destabilizing stimuli, such as a series of internal or external sensory inputs to the body via the somatosensory, vestibular and/or visual systems. These sensory systems can be challenged to trigger automatic postural control via primitive or refined reflexes and pathways. However, only about 5% of people whom we have tested using balance-based, torso-weighting, (BBTW), have high postural control. In a series of BBTW tests described in: U.S. Pat. Nos. 8,215,773; 7,156,792; 7,708,673; US Patent Publications 20110043755; 20100248915; 20070099775; and 20040147377, incorporated herein by reference, we often found that even relatively healthy individuals still had areas of postural insufficiencies that can be improved with the use of strategically placed weights on the body. In addition we found that animals and individuals respond and can move in more coordinated patterns with improved stability from the use of a selective strategically placed weight or weights on the body according to directional and symmetry related tests.

In addition, in some cases we have found that people with directional balance impairment and rotational asymmetry, once corrected, clinically respond positively with improved visual testing, oculmotilities, fixation disparity, steriognosis, depth perception, eye movement, eye alignment, and vestibular ocular coordination. Clinically this has been shown in patients with MS, ataxia, stroke, traumatic brain injury and cerebral palsy. Some people also demonstrate improved coordination testing in the upper (including hand writing, hand to mouth, finger to nose, etc) and lower extremities as well as the trunk over the lower extremities. Some show less torso and extremity tremor as seen in the Lift lab9 (http://www.liftlabsdesign.com/blog/products/2013/01/05/tremor-measurement-and-monitoring/). Individuals also may show improved somato-sensory, visual, and vestibular organization testing on the Smart BALANCE MASTER®, a trademark of NeuroCom, Clackamas, Oregon, for an apparatus, which provides objective assessment and retraining of the sensory and voluntary motor control of balance with visual biofeedback on either a stable or unstable support surface and in a stable or dynamic visual environment. The BALANCE MASTER® system utilizes a dynamic 18″×18″ dual force plate with rotation capabilities to measure the vertical forces exerted by the patient's feet and a moveable visual surround. Body Sway in eyes open and closed and on flat surface and foam and walking have also been shown to improve using inertial measurement units (IMUs) such as APDMTM wearable sensors made by APDM Inc. of Portland, Oreg., which are wireless inertial measurement units about the size of a watch using solid state MEMS technology to precisely record movement with a complete kinematic sensors suite that include triaxial accelerometers, gyroscopes, and magnetometers. In the medical field, these IMUs are ideal for monitoring human movement for clinical research, biomechanical research, physical therapy research, movement disorders research, and athletic tuning.

Patients have also demonstrated improved sensory motor strategies, center of gravity alignment, and motor control. Previously as set forth in U.S. Pat. Nos. 7,156,792; 7,708,763; 8,215,773, and U.S. patent application Ser. No. 12/728,873, incorporated herein by reference, we used a weighting garment and orthotics to improve balance. In particular, the person was put through a series of both static and dynamic tests to determine directional impairment. Adjustable balance evaluation systems were used to determine the strategic placement of a weight(s) to reduce the patient's directional balance loss. For an example of static application, if a person was falling backward and unable to sit or stand, weights could be applied anteriorly until static ability was achieved. In the example of a dynamic application, if a person had the following problems: decreased ability to resist right rotation of the upper torso; loss of balance of the upper torso to the right; and a posterior loss of balance of the upper and lower body, the weighting process would be as follows. A fraction of a pound, e.g. ¼, ½ pound or less, weight would be placed or attached to a garment at a position between the shoulder blade on the left between the spine and scapula. This placement immediately corrects rotation 99% of the time (occasionally the weight is moved up or down a traction of an inch, e.g. ⅛-½ of an inch, to get the correct motor control point for the individual patient where it controls this rotational asymmetry, firing latency, and strength). The person is then perturbed to the right by an exterior force applied to the patient at the upper and lower torso. The rotation weight may correct a lateral imbalance as well. If it did not, the clinician would compare the posterior loss of balance as well as the lateral. If the person lost their balance posteriorly and laterally to the right, then another weight is placed anteriorly and laterally to the left of the navel. In some cases the weight is placed on the same side and the person is weighted into the direction of their loss and it corrects it. Repeated external perturbations and weight adjustments are made until all loss of balance is stabilized. After these adjustments if there is directional loss that can be identified during dynamic or static activities such as walking, standing, standing on foam eyes closed, standing in sharpened Romberg (tandem stance ) etc. minor adjustment in the weight placement or amounts may or may wot be required. Once the individual weighting strategy is determined with the evaluation system, the evaluation is used to create customized garments for the specific patient for enhancing or improving balance.

Described below are customized garments for enhancing balance. Methods of creating customized garments are disclosed, which methods enhance balance using the adjustable balance evaluations.

While the average person or animal doesn't notice that they are not completely in balance, they may note a loss or lack of balance if their balance is challenged in a manner similar to how athletes challenge themselves. It is possible that the personal small amounts of instability lead to subtle imbalances which in turn leave a person at a less than desirable coordination level or mildly off balance using one side of their body more than the other. For example, a person, who is inherently off balance by a small amount, e.g. lean or land on the Lower extremity even a fraction of a pound or heavier. Any imbalance could cause a balance-based arthritis pain or increased muscular tension or pain on one side of their body. This imbalance may lead to sports injuries or inefficiencies in their ability. For example when moving at greater speeds the instability may increase asvelocities, magnitudes, and forces on the body increase. This imbalance may cause decreased efficiency of movement thereby decreasing stamina. In A recent study demonstrated a significant difference in gait velocity, in subjects with MS and healthy controls when strategically placed weights corrected imbalance with the BBTW Method; each group increased averaged its average gait speed by 4%. In addition other spatial temporal aspects of gait improved. (submitted for publication) Patients with MS, vestibular, ataxia and other diagnoses often report they feel more grounded.

While the details of the underlying mechanism, neurology or physiology relating to the efficacy of weight balance mediation, is not well understood, it is appreciated by the inventors of the present disclosure that interaction with the full spectrum of proprioceptive skin and body balance sensors in combination with brain function is intimately involved. In a sense it is re-weighting the sensory system with correct input. The sensory system has multiple sensors that pick up a variety of inputs and relay them in different pathways in the nervous system. There is a redundancy built into the nervous system so that increased input from one source may be picked up by another part of the nervous system enabling better balance and movement control. In addition just as medicines have complementary effects when used in combination, the sensory motor system may also respond to a variety of signals alone or in combination providing enhancement to aspects of balance and motor control. This includes combinations of pharmaceuticals that also increase sensory system and motor output in combination with sensory enhancement in the form of non-weight or weight stimulus or any combinations thereof that can be sensed by or input into the nervous system.

Therefore, what is needed is a method and apparatus in which the mediation of limitations or defects in the proprioceptive skin and body sensors in combination with brain function can be realized or efficaciously practiced using any one of or multiple modal stimuli gateways and any one of or multiple body feed forward or feedback or response pathways.

### BRIEF SUMMARY OF THE INVENTION

The illustrated embodiments of the invention include any type of stimuli needed to maintain the body upright in a static position or along a continuum of more difficult postural control activities depending on the functional level of the subject being tested, making the test harder by increasing the challenge to a person maintaining stability to identify a directional instability by using perturbing stimulus in combination with any type of observation of any type of response to apply any type of non-weight corrective stimulus. We also include any type of perturbing stimulus with any type of observation of any type of response to apply any type of non-weight and weight corrective stimulus. The corrective stimulus in both situations may be any type of non-weight and/or w eight stimulus. The combination of weight and TENS applied to the same nerve pathway controlling the body was better than weight-based stimulus alone or TENS stimulus alone with respect to motor control of a patient with a brain stem bleed. The combination improved the smoothness of the motor control of gait and trunk stability. The definitions of non-weight and weight stimuli for the purposes of this specification and claims are provided below in the disclosure of the detailed embodiments.

The illustrated embodiments in particular include corrective electrical stimulus (called eStim) and/or corrective vibrational stimulus (tactile stimulus). Still further, the invention includes within its scope a stimulating device containing sensors, such as tri-axial accelerometers, gyroscopes and magnetometers, wherein the stimulating device is a weight, a vibrotactile device, and/or an eStim applicator, TENS, using ac/dc current. Any device capable of generating a body or skin stimulus may also have the above sensors and/or specific types of stimulating devices included within it. The stimulating device is capable of attaching to the person via a gel pad, a weighted gel pad, or a vibrotactile stimulator in a gel pad. The stimulating device includes any combination eStim, weight and/or vibration stimulor attached by possibly thermalized gel pad of any weight or any other combination of stimuli with a possibly thermalized gel pad or any form of attachment in or on a garment or device or affixed to the skin in any form.

The illustrated embodiments of the invention include a method of improving a subject's sensory motor system, visual, somato-sensory, and/or vestibular system, comprising the steps of: evaluating the subject's somatosensory, visual, vestibular, and sensory motor nervous system including rotational symmetry, balance reactions in static, dynamic and perturbed stimulation from any position; stimulating the subject's somatosensory, visual, vestibular, muscular balance, and/or motor nerve system by providing one or more selectively placed repositionable non-weight stimuli from a device worn or placed on the subject by any means; reevaluating the subject's somatosensory, visual, vestibular, muscular balance, and/or motor nerve system; comparing the subject's reevaluated somatosensory, visual, vestibular, muscular balance, and/or motor nerve system to the subject's evaluated somatosensory, visual, vestibular, muscular balance, and/or motor nerve system; and repeating the steps of evaluating, stimulating, reevaluating and comparing until the subject's reevaluated somatosensory, visual, vestibular, muscular balance, and/or motor nerve system shows improvement after the comparison of the subject's reevaluated somatosensory, visual, vestibular, muscular balance, and/or motor nerve system to the subject's evaluated somatosensory, visual, vestibular, muscular balance, and/or motor nerve system. The somatosensory, visual, vestibular, muscular balance, and/or motor nerve system can be the object of the steps in any combination or separately according to subject's need.

The repositionable stimuli can be any kind of stimuli which can be sensed, either at subthreshold insensate levels or at perceived above threshold levels. Devices which are capable of producing such stimuli include the output from at least one or more electrical electrodes, pressure transducers, vibration transducers, acoustic transducers, stretch transducer, aromatic generators, light sources, flavor sources, thermal transducers, pain generators, or stimuli generator.

The non-weight stimuli of the illustrated embodiment are electrical skin stimuli (eStim) produced by transcutaneous electrical nerve stimulation (TENS) devices and/or skin tactile stimuli produced by vibrotactile stimuli. TENS is the use of electric current produced by a device to stimulate the nerves for therapeutic purposes. TENS by definition covers the complete range of transcutaneously applied currents used for nerve excitation, although the term is often used with a more restrictive intent, namely to describe the kind of pulses produced by portable stimulators used to treat pain, no such restrictive intent is included in the current specification. The TENS device is usually connected to the skin using two or more electrodes. A typical battery-operated TENS device is able to modulate pulse width, frequency and intensity. Generally, TENS is applied at high frequency (>50 Hz) with an intensity below motor contraction (sensory intensity) or at a low frequency (<10 Hz) with an intensity that produces motor contraction.

In another embodiment the method of improving a subject's vestibular, muscular balance, and/or neurosensory motor nerve system comprises the steps of evaluating the subject's vestibular, muscular balance, and/or motor nerve system to determine a probable cause of limitation or defect therein; and stimulating the subject's vestibular, muscular balance, and/or motor nerve system by attaching at least one or more repositionable non-weight stimuli to a device worn or attached to the subject by any means according to the probable cause of limitation or defect therein.

The method further includes the steps of: reevaluating the subject's vestibular, muscular balance, and/or neurosensory-motor nerve system; comparing the subject's reevaluated vestibular, muscular balance, and/or motor nerve system to the subject's evaluated vestibular, muscular balance, and/or motor nerve system; and repeating the steps of evaluating, stimulating, reevaluating and comparing until the subject's reevaluated vestibular, muscular balance, and/or neurosensory motor nerve system shows improvement after the comparison of the subject's reevaluated vestibular, muscular balance, and/or motor nerve system to the subject's evaluated vestibular, muscular balance, and/or motor nerve system.

The illustrated embodiments also include any apparatus used in the performance of any of the above methods. For example, the apparatus comprises: a pair of eyeglasses or an eyeglass frame through which visual stimuli would be provided; a garment or wearable device through which nonvisual, non-weight stimuli would be provided, or a leg, arm, hand or foot prosthesis. The garment is configured to be worn on the subject's torso or body or the garment is a cap, headband, or hat or neck brace. In another embodiment the apparatus includes: an orthotic configured to fit the subject's torso, including a bra, belt, a strip of magnetic material to a piece of a garment or apparatus; or a neuroprosthesis, worn or attached anywhere on the body. The orthotic would provide a sense of confinement, support or rigidity for a body part. The prosthesis is a prosthetic limb or more specifically a prosthetic leg, which would provide a sense of bodily extension or replacement for a lost limb. Such orthotics or prostheses provide at least corrective stimuli or provide an attachment platform or carrier through which eStim or vibrational corrective stimuli can be provided. A prosthetic is anything that replaces a body organ or a portion of one and replaces its functions. A heating aid is a prosthetic in this sense of the term. A sensory system is one of the body organ systems, like the auditory system as a whole, which would include the hearing aid.

More particularly, the illustrated embodiments of the invention include A method of improving functioning of at least a portion of a subject's nervous pathway system including the steps of: observing the subject's ability to maintain their stability in an unstimulated state; selectively applying a non-weight stimulus to the on the subject to enhance the subject's stability; observing the subject's ability to maintain their stability with the selectively applied non-weight stimulus; and repeating the steps of selectively applying a non-weight stimulus to the on the subject to enhance the subject's stability and observing the subject's ability to maintain their stability with the selectively applied non-weight stimulus until an improvement in stability control by the subject is observed.

The illustrated embodiments further include a method combining the above steps with those that follow below, omitting or limiting the number of repetitions of the above steps before performing the steps that follow below, or omitting all of the above steps and performing the steps of: stimulating at least the portion of the subject's nervous pathway system by providing at least one perturbing stimulus; evaluating the subject's response to the perturbing stimulus; providing at least one corrective non-weight stimulus to the subject's nervous pathway system; restimulating at least the portion of the subject's nervous pathway system by providing the at least one perturbing stimulus; comparing the subject's response to the restimulation of at least the portion of the subject's nervous pathway system; and repeating the steps of stimulating, evaluating, providing at least one corrective non-weight stimulus, providing at least one corrective non-weight stimulus, and comparing the subject's response to the restimulation until the subject's response improves to an optimal one for the treated subject.

The step of providing at least one corrective non-weight stimulus to the subject's nervous pathway system comprises providing at least one corrective non-weight and weight stimulus in combination to the subject's nervous pathway system.

The step of repeating the steps of stimulating, evaluating, providing at least one corrective non-weight stimulus, providing at least one corrective non-weight stimulus, and comparing the subject's response to the restimulation until the subject's response improves to an optimal one for the treated subject comprises providing at least one corrective non-weight stimulus to a different location on the subject, providing a different degree, amount or intensity of the at least one corrective non-weight stimulus to the subject at the same or different location on the subject, or providing a different kind of corrective non-weight stimulus to the subject at the same or different location on the subject or any combination thereof.

The step of providing at least one corrective non-weight stimulus to a different location on the subject, providing a different degree, amount or intensity of the at least one corrective non-weight stimulus to the subject at the same or different location on the subject, or providing a different kind of corrective non-weight stimulus to the subject at the same or different location on the subject further comprises providing at least one corrective weight stimulus in combination with the non-weight stimulus.

The step of providing at least one corrective non-weight stimulus to the subject's nervous pathway system comprises providing a stimulus from an from one or more electrical stimulators, pressure transducers, vibration transducers, acoustic transducers, aromatic generators, light sources, flavor sources, thermal transducers, pain generators, or bodily stimuli generator.

The steps of stimulating, evaluating, providing at least one corrective non-weight stimulus, providing at least one corrective non-weight stimulus, and comparing the subject's response to the restimulation of the subject's nervous pathway system comprises stimulating, evaluating, providing at least one corrective non-weight stimulus, providing at least one corrective non-weight stimulus, and comparing the subject's response to the restimulation of the subject's vestibular, vision, muscular balance, sensory motor nervous, proprioceptive or somatosensory systems.

The illustrated embodiments also include an apparatus for improving functioning of at least a portion of a subject's nervous pathway system including a garment or device worn or disposed on the subject; and at least one stimulator disposed on, in or within the garment or device capable of providing at least one corrective non-weight stimulus to the subject's nervous pathway system. The kind of non-weight stimulus provided by the stimulator to the subject's nervous pathway system, the amount, degree or intensity of the stimulus provided by the stimulator to the subject's nervous pathway system, and/or where the body location of non-weight stimulus provided by the stimulator to the subject's nervous pathway system is determined by any one of the reiterative diagnostic methods described above or any other reiterative diagnostic method now known or later devised for determining the application of a type, degree and location of a non-weight stimulus to the subject with or without a weight stimulus.

In one embodiment the device comprises a pair of eyeglasses or an eyeglass frame.

In another embodiment the garment is configured to be worn on the subject's body, head or torso. The garment includes a cap, headband, hat or neck brace.

In still another embodiment the device comprises an orthotic configured to fit the subject's torso, including a bra, belt, vest, undergarment or a strip of magnetic material to a piece of a garment.

In yet another embodiment the device comprises a prosthesis worn or attached to or on the subject's body. The prosthesis comprises a prosthetic limb or a prosthetic leg.

The illustrated embodiment include not only the end stage product with or without indicia to be worn or disposed on the subject but also a test garment or test device having indicia thereon used by the therapist in the assessment method and to which the at least one stimulator is attachable at any location thereon to allow for reiterative repositioning of the stimulator thereon and recordal of an optimal location of attachment for the treated subject.

The apparatus further includes at least one other stimulator disposed on, in or within the garment or device capable of providing at least one corrective weight stimulus to the subject's nervous pathway system in combination with the at least one stimulator capable of providing at least one corrective non-weight stimulus.

The apparatus further includes a plurality of stimulators capable of providing corresponding corrective weight stimuli to the subject's nervous pathway system in combination with a plurality of stimulators capable of providing at corresponding corrective non-weight stimuli.

The stimulator comprises an eStim stimulator, or a vibrotactile stimulator.

In another embodiment tire stimulator comprises a combined non-weight eStim and vibrotactile stimulator for providing corresponding stimuli at the same location on the body of the subject.

In yet another embodiment the stimulator comprises a combined non-weight eStim and vibrotactile stimulator and a weight for providing corresponding stimuli at the same location on the body of the subject.

An apparatus for improving functioning of at least a portion of a subject's nervous pathway system including a garment or device worn or disposed on the subject; an electrical stimulator disposed on, in or within the garment or device capable of providing a corrective electrical stimulus to the subject's nervous pathway system through a skin-contact electrode; and a corrective weight combined with the electrical stimulator to provide a weight-based stimulus. The combined electrical stimulator or skin-contact electrode and weight could be supplied without disposition in, on or within the garment or device, but be directly applied to the body location and with a degree of stimuli as determined by the reiterative diagnostic method.

The apparatus includes a stimulator which provides:

a. A non-weight stimulus that is magnetic and is combined with a weight stimulus.
b. A non-weight stimulus that is vibration and is combined with a weight stimulus.
c. A non-weight stimulus that is vibration and is combined with an electrical stimulus.
d. Non-weight stimuli that are magnetic and electrical and are combined with a weight stimulus.
e. A non-weight stimulus that is a tensile force and is combined with a weight stimulus.
f. A non-weight stimulus that is a tensile force and is combined with vibration.
g. A non-weight stimulus that is a tensile force and is combined with an electrical stimulus.
h. A non-weight stimulus that is pressure and is combined with a weight or non-weight weight stimulus.
i. A non-weight stimulus that is thermal and is combined with a weight or non-weight stimulus.
j. Any non-weight stimulus that the sensory system can pick up either alone or in any combination.

### BRIEF DESCRIPTION OF THE DRAWINGS

FIG. 1 is a diagram of a combined or integrated package for an eStim, vibrotactile stimulator and weight for use in one embodiment of the invention.

### DETAILED DESCRIPTION OF THE PREFERRED EMBODIMENTS

As described above strategic weight placement provides a sensory input recognized by the animal or person that increases physical stability. While weight placement originally began as an observation and reiterative test-retest process with each weight placement on the person until balance control or stability in orientation, verticality, rotation resistance to perturbation as measured by direction, magnitude and latency was improved, in time, a correlation was made between an observation of a specific weight placement and improved control of the patients' particular balance problems, and muscle, nerve or biofunction related to balance and the correct weight(s)and placement(s). Some patients also report decreased pain and effects on urinary flow, cognitive dual tasking such as walking and performing math or spelling. In addition the sensory re-weighting also improves how the visual and vestibular system is responding in to Neurocom Sensory organizational testing and is related to decreased falls in during subject testing.

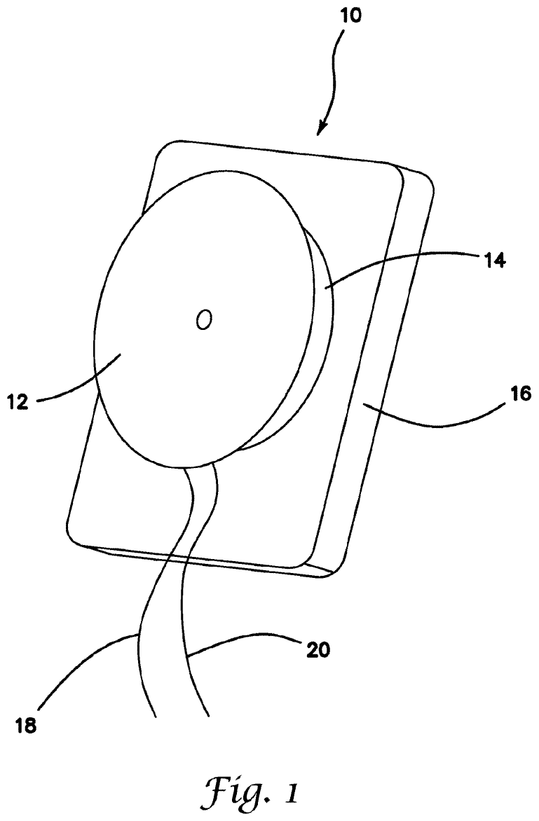

In addition to the weight placement mediation discussed above, other tests and stimuli are applied to the body as disclosed below even where stimuli through weighting may or may not be beneficial. Strategic placement of sub-threshold stimulation, threshold stimulation, or contractile or tensile forces applied to the skin, muscles or tissue, taping, strapping, vibrating, touching, scratching, thermal stimulus (temperature), electrical stimulation, optical stimulation, diathermic stimulation, pressure, suction, acupressure or acupuncture or any other kind of stimulation of the proprioceptive, visual, and/or vestibular system body sensors in combination with brain or central nervous system functions can be employed in an analogous manner by a generalization of the iterative test/retest weight placement protocols an added to by other perturbation methods where one identifies the directional loss of balance. When the visual, vestibular system is perturbed, a harder paradigm is created for the bodily control system to handle, so that the dysfunction, which could be uncovered includes not only directional control but symptomatic reactions such as dizziness or nausea. However, it is to be understood that the illustrated embodiments of the invention am not limited to a method, which only emulates the prior procedures for strategic weight placement tor balance remediation, but contemplates entirely new methodologies and apparatus as may be based on different biophysical stimuli and modernisms. For example, included is stimuli using an electrical electrode muscle stimulation (eStim), including but not limited to AC, DC, pulsed or otherwise modulated signals through contact electrodes or electromagnetic coupling into or on the body. Expressly contemplated is any modulation or pulsation of any electrical stimulus at any frequency or frequencies, pulse widths, amplitudes or phases or combinations of the same. The electrode placement could be identical or similar to weight placement patterns or could be determined on a patient-by-patient basis using any kind of bioelectrical (e.g. electromyogram (EMG)) or physical observation or measurement of sensory motor neuromuscular or proprioceptive skin and body sensor responses in combination with brain function to cancel out impairment of muscle or nerve function. Multiple stimuli could be included in a combined stimulator for application to a single body location, e.g. an eStim electrode 12 with its lead 18 coupled to a TEN unit (not shown), vibrotactile stimulator 14 with its lead 20 coupled to a battery pack (not shown) and weight 16 could be combined in an integrated package 10 as shown in FIG. 1 for application to the body at the same location. For instance we could find that in people with disability or prone to injury that their body electromyograph (EMG) shows certain muscles are activatable at sub-thresholds and others are activatable at over-thresholds. Using APDM wearable sensors, i.e. inertial measurement units incorporating wireless accelerometers, gyroscopes, and magnetometers, even subtle bodily motion changes can be observed in response to the stimuli and the stimuli controlled until the desired bodily motion sequence and response is obtained. Rotational and triaxial maladies can respond to each directional loss by applying a stimulus described above to the visual, vestibular or somatosensory at the head, neck, trunk, pelvis or on the extremity to de-rotate or stimulate the malady into a more neutral position thus improving motion via attaching a stimulator having various controllable outputs or a combination of stimulators at different positions, each providing a different or identical type and format of stimulation. For instance stimulation by mechanical vibration and weight can be controllably applied to the body to obtain the desired response(s) The stimulation need not only be applied from external sources, but can also include internally applied stimuli or prescription or nonprescription drugs. For example, nonprescriptive medicaments, such as Neurostim™, which is a nootropic or one of a family of brain-enhancing drugs initially used to relieve symptoms of mind-degenerative illnesses such as Alzheimer's and dementia, made by Mind Nutrition LLC of Santa Fe, N. Mex., can be employed in combination with external stimuli.

It is anticipated that strategic weight placement according to the prior patents cited above may not be identical to electrode or other stimuli placements on the body, although it is expected that some overlap will occur. One object of the invention is to provide the necessary input with the smallest amount of coverage on the body so the wearer and the public will not know the wearer has on a corrective, sensory stimulating orthotic or neuroprosthetic device.

In theory when the body is unable to resist a rotational force, anterior or posterior or lateral perturbation of any part of the body, the force or perturbation could come from the floor or anywhere on the body, an input to the body like a nudge, or a stimulation of the vestibular, sensory or visual system. This creates a physical instability. Cognitive issues may also arise since the brain is having to focus the energy on being upright and cannot perform more than one task. If all forces are not equal and any directional instability is found, we have an opportunity to create positive change by adding input or natural bodily feedback to increase a subthreshold response, decrease an over-stimulated response, or change a co-contraction to more a normal response of agonists and antagonist muscles responses. Our system compares normal responses to those which are abnormal and normalizes the responses.

Recently, we saw that when a patient was not able to resist rotation of the upper torso, there was a lack of muscle firing or activation on one side of the body and fasciculation on the other side of the body. When we tested the other side, most or many of the muscles engaged. An EMG recorded the differences on both sides of the body. When the weight was strategically placed to correct the rotational response, most of the firing was normal. If we perform the BBTW test with EMG observations, we find subthreshold activation of muscle groups in static as well as in dynamic testing. If we perform other means of perturbing the body with machines and with electromyography (EMG), we find patterns of subthreshold or over-threshold responses. In an EMG test of three patients with low back and radicular pain, we found high firing patterns of paraspinal muscles that were immediately reduced by 50% with BBTW. By comparing the wrong sensory-motor nerve responses with correct sensory motor responses, we are able to facilitate the correct response in certain muscle groups to control the person's body. We can then treat those areas with selected appropriate sensory stimuli to bring them into a normalized response via the neurosensory-motor system.

In this disclosure, we sense what the body response to perturbations or simply selected body movements by any one of various means now known or later devised to determine an impairment and then apply one or more stimuli or inputs that feed the correct information to the body's natural sensory neuromotor system to create a feed forward and better feedback loop to the brain and/or central nervous system. We use tests to find impairments in the postural control system and add sensory information singularly or in combination to mediate the identified impairment. Our method identifies problems with static or dynamic control through observations and/or perturbations or methods challenging the input to various sensory systems, then add a sensory input to the same or different or in combination sensory motor system to correct the dysfunction followed by a reiterative test/retest correction(s) to stabilize and improve or mediate body balance or other functions.

Although it is not clear how the mediation functions within each patient, the disclosed method and use of the disclosed apparatus shows material changes or improvements in the somatosensory, visual and vestibular organization resulting in improved balance, vision, coordination, motor control, writing, speech, swallowing and other cognitive areas. In some cases, the mediation, improvement or restoration of normal muscle and nerve function is surprising, unexpected, unpredicted and startling, even though it is repeatable in the patient. The same mediation does not, however, work equally well or in the same way in all patients or to the same degree. The nature and degree of impairment in the functioning of proprioceptive skin and body sensors in combination with brain or central nervous system functioning is highly variable from one patient to the next, particularly given the wide variety and multiplicity of causative agents or mechanisms that could cause such impairments. The effects of the mediation can be temporary or long term, which appears to be related to re-teaching the brain or central nervous system to subconsciously control muscular function using a different neurosensory motor protocol.

Therefore, it must be understood that the illustrated embodiments of the invention include a device or devices that detects the abnormal neurosensory functioning of proprioceptive skin and body sensors, vision, vestibular or somatosensory, and/or motor sensors in combination with brain or central nervous system functioning; and a method or device that provides a mediating stimulus or stimuli to the proprioceptive skin and body sensors in combination with brain or central nervous system. The functioning of a proprioceptive skin and body sensor in combination with brain or central nervous system functioning is again tested to determine whether any normalization of functioning has occurred. If not, or if the observed functioning of a proprioceptive skin and body sensor in combination with brain or central nervous system functioning is not improved, a different stimulus or stimuli is provided or a different location or input location is provided for the stimulus or stimuli. The process and use of the devices continues reiteratively until satisfactory mediation is observed.

Any device or methodology now known or later devised for stimulating a response from a proprioceptive skin and/or body sensor in combination with brain or central nervous system may be employed. For example, a visual stimulation such as used in virtual reality training could be introduced to cause imbalance as a sensory perturbation. We would then alter the directional impairment with a corrective input for stabilization utilizing oilier areas of the sensory system to stabilize the visual induced impairment.

Any device or methodology now known or later devised for detecting the response of a proprioceptive skin and/or body sensor in combination with brain or central nervous system may be employed. The method disclosed above may employ any device for creating a response from a proprioceptive skin and/or body sensor in combination with brain or central nervous system in combination with any device for detecting a response from a proprioceptive skin and/or body sensor in combination with brain or central nervous system. The sources of stimuli as well as the sensors of the responses may be applied directly to the body or held in contact with or proximity to the body or any portion thereof by any device, garment, appliance or other means for positioning, holding, and/or allowing for repositioning of such sources and sensors.

In practice certain categories of impairment are found to be responsive to certain stimulus or stimuli and certain placements of the stimulus or stimuli. As the practitioner gains experience in diagnosing the impairment and the effective mediation for each category of impairment, the reiterative process becomes more directed and efficacious. For example, in the case of impairments related to balance control of the torso, use of the apparatus, namely small body weights strategically attached to a Velcro® receivable garment, has led to an experiential knowledge database, that can be taught to others, that impairment in one or more directions corresponding to muscle groups or to a portion of the proprioceptive system in the torso is often effectively mediated by weight placed on or in the location of the opposite on the torso on the muscle or at least to the overlying skin positions.

Therefore, as another example, instead of stimulation by placement of small body weights, the attachment of selected subthreshold or threshold electrical, vibratory muscle activations of the torso skin, muscle groups by transcutaneous electrical nerve stimulation (TENS) or muscle stimulators using different signal protocols are used. In another example, selective stimulation of different skin proprioceptive sensors, e.g. superficial, dermal, or subdermal tactile, thermal, pressure, or pain sensors would be stimulated using any modality effective for activating these sensors. For example, a small lightweight patch of small nonpenetrating or micropenetrating spikes, like a miniature bed of nails, pressed against the skin using a tight fitting, compression garment or device, is used in place of the small weight placement discussed above. Implantable electrodes or biodevices can be employed.

Instead of observing the responses to the stimuli by visual observation by a skilled practitioner in response to gross anatomical body perturbation forces, computer analysis and/or observation of EMG sequences and traces, similar to using electrocardiograms for cardiovascular diagnosis, are employed.

The responses of the brain and any selected part of the nervous system may be detected in the above modality by any modality now known or later devised, include functional MRI of any body pan or electroencephalogram (EEG) of brain or electrical trace of CNS function. According to the spirit and scope of the invention, the number and variety of stimuli that can be applied, their combination, their sequencing and formatting is nearly limitless. Similarly, the number and variety of sensing or observational modalities that can be applied, their combination, their sequencing and formatting is also nearly limitless.

However, for the purposes of this disclosure and clarity of terminology in the following claims, the term, “non-weight stimulus” and similar terms, e.g. “non-weight-based stimulus”, is defined to include all forms and sources of stimuli perceivable by vision, smell, taste, sound or touch, and any equivalents thereto later discovered or developed, other than stimuli which arise from the use of a static mass or weight applied directly or indirectly to the body or skin, including but not limited to a garment, appliance or device for coupling such a mass or weight to the body or skin. Thus, “non-weight stimulus” is to be understood as not including any apparatus or methodology disclosed or claimed in any of the U.S. Pat. Nos. 7,156,792; 7,708,763; 8,215,773, or U.S. patent application Ser. No. 12/728,873, or in any divisional, continuation or currently filed continuation-part application related to the same under 35 USC 119, 120. Correspondingly, the term “weight stimulus” and similar terms, is defined to include stimuli which arise from the use of a static mass or weight applied directly or indirectly to the body or skin, including but not limited to a garment, appliance or device for coupling such a mass or weight to the body or skin. Thus, “weight stimuli” or “weight-based stimulus” is to be understood as including any apparatus or methodology disclosed or claimed in any of the U.S. Pat. Nos. 7,156,792; 7,708,763; 8,215,773, or U.S. patent application Ser. No. 12/728,873, or in any divisional, continuation or currently filed continuation-part application related to the same under 35 USC 119, 120.

It is expressly understood that the entirety of the disclosure of U.S. Pat. No. 8,215,773 (hereinafter ‘773 patent) has been and is incorporated into the present specification by reference. Therefore, the illustrated embodiments of the present invention is incorporated into and combined in all of the embodiments in the ‘773 patent. It is to be expressly understood that any mechanism for attachment of the devices into or on the garments or devices of the ‘773 patent is contemplated. For example, the entirety of the garment or worn device may be Velcro receivable so that the perturbing stimulator and/or corrective stimulator may be mounted, attached, positioned or disposed on the outside or inside surface of the garment or worn device or even within the garment or worn device at any, all or some positions or at a selected position or region on, in or within the garment or worn device. The temporary or permanent affixation on, in or within the garment or worn device may be by any mechanism or means now known or later devised, including but not limited to hook-latch, adhesive, welding, gluing, sewn, magnetic, riveted, pinned, bolted, belted, tied, strapped, laced, bound, snapped, connected, joined, any type of mechanical or electrostatic attachment, and/or mechanical compression by the garment or worn device. Accessory devices for recording or sensing responses or movement, or for powering the perturbing or corrective stimuli may be incorporated into the garment or word device, or carried in separately attached packs or belts, it is also contemplated that the garment or worn devices will be combined with wirelessly coupled accessory apparatus used for the disclosed functioning, for example as may be included in wheelchairs, walkers, prostheses, orthotics or other collateral apparatus. Examples of some of the garment or worn device are shown in FIGS. 1-38 of the ‘773 patent and described therein. The various garments and devices in FIGS. 1-38 of the ‘773 patent may be fitted with any one or more of the non-weight and weight stimulators disclosed herein or with the integrated multiply stimuli stimulator of FIG. 1. Thus, it is to be understood that many of the garments and devices with which the stimulators of the disclosed embodiments are intended to be used or may be used are illustrated in the incorporated patents, and in the ‘773 patent in particular. Regardless of how the perturbing stimulus and corrective stimulus is coupled into the patient's sensory or nervous pathways, it is selected and applied as determined by the observational and/or perturbative reiterative diagnostic methodology of the invention.

Each patient or individual is different than each other patient or individual. Therefore, other than in very improbable and limited circumstances will the application of perturbing and corresponding corrective stimuli be exactly identical for any two patients or individuals. Just as each patient or individual has a unique set of fingerprints, so each patient or individual has a unique corrective set of stimuli that is optimal or effective with that patient or individual. Further, as the patient's or individual's brain learns to compensate for any dysfunction using the corrective stimuli, the application of the corrective stimuli may or will change over time. The diagnostically determined corrective stimuli needed may change in time even if there is no disease progression that changes the nervous system pathways, because of brain adaptation.

Further, wherever the term, “body” is used, it is to be understood to include the whole of or any portion of the body, such as the head, torso, limbs, skin, neck, hands, feet, or a sensory organ or sensory system of the body including vision, smell, taste, sound or touch, or any internal portion of the body.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, not withstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

1-31. (canceled)

33. A device for improving functioning of at least a portion of a subject's nervous system pathway, the device comprising:
an electrical stimulation applicator comprising a skin-contact electrode having a gel pad configured to provide a corrective electrical stimulus to the subject's nervous system pathway at a location of skin contacted by the skin-contact electrode; and
a corrective weight combined with the skin contact electrode configured to provide a weight-based stimulus at the location of skin, wherein only the skin contact electrode of the electrical stimulation applicator contacts the subject.

34. The device of claim 33, further comprising an electrical stimulator.

35. The device of claim 34, wherein the electrical stimulator is configured to provide a corrective electrical stimulus having a frequency of 50 Hz or greater, with an intensity below motor contraction.

36. The device of claim 33, wherein the corrective weight is combined with the skin contact electrode by an attachment that is not permanent.

37. The device of claim 36, wherein the attachment is an adhesive or a fastener.

38. The device of claim 33, wherein the corrective weight has a weight of from 1/16 pound to ½ pound.

39. The device of claim 33, wherein the device further comprises at least one sensor comprising an accelerometer, gyroscope, or magnetometer.

40. The device of claim 39, wherein the at least one sensor is wireless.

41. The device of claim 33, wherein the device further comprises a vibration stimulator configured to provide a corrective vibrational stimulus to the subject's nervous system pathway at the location of skin contacted by the skin-contact electrode of the device.

42. The device of claim 41, wherein the vibration stimulator contacts the location of skin via a gel pad.

43. A device for improving functioning of at least a portion of a subject's nervous system pathway, the device comprising:
a vibrotactile stimulator, wherein the vibrotactile stimulator is configured to provide a corrective vibrational stimulus to the subject's nervous system pathway at a location of skin contacted by the vibrotactile stimulator; and
a corrective weight combined with the vibrotactile stimulator configured to provide a weight-based stimulus at the location of skin, wherein only the vibrotactile stimulator contacts the subject.

44. The device of claim 43, wherein the vibrotactile stimulator is attached to the location of skin via a gel pad.

45. The device of claim 43, wherein the corrective weight is combined with the skin contact electrode by an attachment that is not permanent.

46. The device of claim 45, wherein the attachment is an adhesive or a fastener.

47. The device of claim 43, wherein the corrective weight has a weight from 1/16 pound to ½ pound.

48. The device of claim 43, wherein the device further comprises at least one sensor comprising an accelerometer, gyroscope, or magnetometer.

49. The device of claim 48, wherein the at least one sensor is wireless.

50. The device of claim 43, wherein the device further comprises an electrical stimulation applicator comprising a skin-contact electrode configured to provide a corrective electrical stimulus to the subject's nervous system pathway at the location of skin.

51. The device of claim 50, wherein the skin-contact electrode comprises a gel pad.

52. The device of claim 50, further comprising an electrical stimulator, wherein the electrical stimulator is configured to provide a corrective electrical stimulus having a frequency of 50 Hz or greater with an intensity below motor contraction.

53. A device for improving functioning of at least a portion of a subject's nervous system pathway, the device comprising:
a stimulator comprising a gel pad configured to provide a corrective stimulus to the subject's nervous system pathway at a location of skin contacted by the stimulation applicator; and
a corrective weight combined with the stimulator configured to provide a weight-based stimulus at the location of skin, wherein only the stimulator contacts the subject.
